(19) United States
(12) Patent Application Publication
(10) Pub. No.: US 2015/0065345 A1
(43) Pub. Date: Mar. 5, 2015

(54) 1,2,4 - TRIAZOLE DERIVATIVES AS HERBICIDALS

(71) Applicant: SYNGENTA LIMITED, Bracknell (GB)

(72) Inventor: Glynn Mitchell, Bracknell (GB)

(73) Assignee: SYNGENTA LIMITED, Bracknell (GB)

(21) Appl. No.: 14/389,141

(22) PCT Filed: Mar. 27, 2013
(86) PCT No.: PCT/EP2013/056572

§ 371 Date: Sep. 29, 2014

(30) Foreign Application Priority Data
Mar. 29, 2012 (GB) 1205657.8

(51) Int. Cl. A01N 43/653; C07D 401/12; C07D 249/14

(52) U.S. Cl. CPC A01N 43/653; C07D 249/14; C07D 401/12
USPC 504/103; 548/265.4; 504/273; 504/139; 546/272.4; 504/253; 504/130

(57) ABSTRACT

The present invention relates to compounds of Formula (I), or an agronomically acceptable salt of said compounds wherein R¹, R², X, R⁴, R⁵ and R⁷ are as defined herein. The invention further relates to herbicidal compositions which comprise a compound of Formula (I), and to their use for controlling weeds, in particular in crops of useful plants.

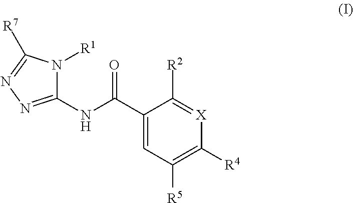

The present invention relates to novel herbicidal compounds, to processes for their preparation, to herbicidal compositions which comprise the novel compounds, and to their use for controlling weeds, in particular in crops of useful plants, or for inhibiting plant growth.

Herbicidal N-(Tetrazol-5-yl) and N-(Triazol-5-yl) arylcarboxamides are known from WO2012/028579. The present invention relates to the provision of further herbicidal triazolyl compounds.

Thus, according to the present invention there is provided a compound of Formula (I):

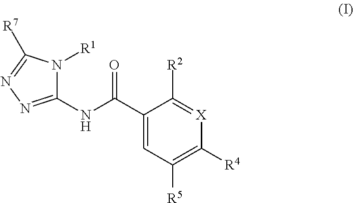

or an agronomically acceptable salt thereof,

wherein:—
R¹ and R⁷ are independently selected from the group consisting of hydrogen, C₁-C₆ alkyl, C₁-C₆ haloalkyl and C₁-C₆alkoxy-C₁-C₃alkyl;
or together R¹ and R⁷ form a C₁-C₆alkylene chain, a C₁-C₆haloalkylene chain or a C₁-C₃alkyleneoxy-C₁-C₃alkylene chain;
R² is selected from the group consisting of C₁-C₆ alkyl-, C₁-C₆ haloalkyl-, C₁-C₆ alkoxy-C₁-C₆alkyl-, C₁-C₃alkoxy-C₂-C₃alkoxy-C₁-C₃alkyl-, halogen, cyano, nitro, C₁-C₆alkyl-S(O)p- and C₁-C₆haloalkyl-S(O)ₚ—;
X is CR³ or N;
R³ is selected from the group consisting of hydrogen, halo, C₁-C₆alkyl, C₁-C₆halo alkyl, C₁-C₆alkoxy-C₁-C₆alkyl, C₁-C₆haloalkoxy-C₁-C₆alkyl, C₁-C₆alkoxy-C₁-C₆alkoxy-C₁-C₆alkyl, C₁-C₆alkoxy, C₁-C₆haloalkoxy, C₁-C₆alkoxy-C₁-C₆alkoxy, C₁-C₆alkylamino, C₁-C₆dialkylamino-, piperidino, morpholino, cyano, C₁-C₆alkyl-S(O)p- and C₁-C₆haloalkyl-S(O)ₚ—;
R⁴ is selected from the group consisting of hydrogen, C₁-C₆alkyl, C₁-C₆haloalkyl, C₁-C₆alkoxy-C₁-C₆alkyl, halo, cyano, nitro, C₁-C₆alkyl-S(O)p- and C₁-C₆haloalkyl-S(O)ₚ—;
or R³ and R⁴ together form a saturated 5- or 6-membered ring, optionally containing an oxygen or a S(O)ₚ heteroatom, the 5- or 6-membered ring being optionally substituted by one or more R⁶,
R⁵ is selected from the group consisting of, hydrogen, halogen, C₁-C₆ alkyl and C₁-C₆ haloalkyl;
or R⁴ and R⁵ together form a 5- or 6-membered aromatic ring, optionally containing a nitrogen heteroatom, the 5- or 6-membered aromatic ring being optionally substituted by one or more R⁶;
R⁶ is selected from the group consisting of halo, C₁-C₆alkyl, C₁-C₆haloalkyl, C₁-C₆alkoxy-C₁-C₆alkyl, C₁-C₆haloalkoxy-C₁-C₆alkyl, C₁-C₆alkoxy-C₁-C₆alkoxy-C₁-C₆alkyl-, C₁-C₆alkoxy and C₁-C₆haloalkoxy; and
p=0, 1 or 2.

Halogen (or halo) encompasses fluorine, chlorine, bromine or iodine. The same correspondingly applies to halogen in the context of other definitions, such as haloalkyl or halophenyl.

Haloalkyl groups having a chain length of from 1 to 6 carbon atoms are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl, heptafluoro-n-propyl and perfluoro-n-hexyl.

Alkoxy groups preferably have a chain length of from 1 to 6 carbon atoms. Alkoxy is, for example, methoxy, ethoxy, propoxy, isopropoxy, n-butoxy, isobutoxy, sec-butoxy or tert-butoxy or a pentyloxy or hexyloxy isomer, preferably methoxy and ethoxy. It should also be appreciated that two alkoxy substituents present on the same carbon atom may may be joined to form a spiro group. Thus, the methyl groups present in two methoxy substituents may be joined to form a spiro 1,3 dioxolane substituent, for example. Such a possibility is within the scope of the present invention.

Haloalkoxy is, for example, fluoromethoxy, difluoromethoxy, trifluoromethoxy, 2,2,2-trifluoroethoxy, 1,1,2,2-tetrafluoroethoxy, 2-fluoroethoxy, 2-chloro ethoxy, 2,2-difluoroethoxy or 2,2,2-trichloroethoxy, preferably difluoromethoxy, 2-chloroethoxy or trifluoromethoxy.

C₁-C₆alkyl-S-(alkylthio) is, for example, methylthio, ethylthio, propylthio, isopropylthio, n-butylthio, isobutylthio, sec-butylthio or tert-butylthio, preferably methylthio or ethylthio.

C₁-C₆alkyl-S(O)— (alkylsulfinyl) is, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₆alkyl-S(O)₂— (alkylsulfonyl) is, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

Alkylamino is, for example, methylamino, ethylamino, n-propylamino, isopropylamino or a butylamino isomer. Dialkylamino is, for example, dimethylamino, methylethylamino, diethylamino, n-propylmethylamino, dibutylamino or diisopropylamino. Preference is given to alkylamino groups having a chain length of from 1 to 4 carbon atoms.

Alkoxyalkyl groups preferably have from 1 to 6 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

In a preferred embodiment of the invention R¹ is methyl or ethyl.

In another embodiment R⁷ is selected from the group consisting hydrogen, methyl and ethyl.

In another embodiment of the present invention R² is selected from the group consisting of methyl, fluoro, chloro, nitro, methoxyethoxymethyl-, trifluoromethyl and methyl-S(O)₂—.

In another embodiment of the present invention R² is selected from the group consisting of methyl, fluoro, chloro, trifluoromethyl and methyl-S(O)₂—.

In another preferred embodiment of the present invention, X is CR³.

In another preferred embodiment of the present invention, X is CR³ and R³ is hydrogen or CF₃CH₂OCH₂—.

In another embodiment, R⁴ is selected from the group consisting of hydrogen, trifluoromethyl, fluorine, chlorine and methyl-S(O)₂—.

In another embodiment, R⁴ is selected from the group consisting of hydrogen, trifluoromethyl and methyl-S(O)₂—.

In another embodiment, R⁵ is selected from the group consisting of hydrogen, fluorine, chlorine, bromine and methyl.

Compounds of Formula I may contain asymmetric centres and may be present as a single enantiomer, pairs of enantiomers in any proportion or, where more than one asymmetric centre are present, contain diastereoisomers in all possible ratios. Typically one of the enantiomers has enhanced biological activity compared to the other possibilities.

Similarly, where there are disubstituted alkenes, these may be present in E or Z form or as mixtures of both in any proportion.

Furthermore, compounds of Formula I may be in equilibrium with alternative tautomeric forms. It should be appreciated that all tautomeric forms (single tautomer or mixtures thereof), racemic mixtures and single isomers are included within the scope of the present invention.

The present invention also includes agronomically acceptable salts that the compounds of Formula I may form with amines (for example ammonia, dimethylamine and triethylamine), alkali metal and alkaline earth metal bases or quaternary ammonium bases. Among the alkali metal and alkaline earth metal hydroxides, oxides, alkoxides and hydrogen carbonates and carbonates used as salt formers, emphasis is to be given to the hydroxides, alkoxides, oxides and carbonates of lithium, sodium, potassium, magnesium and calcium, but especially those of sodium, magnesium and calcium. The corresponding trimethylsulfonium salt may also be used.

The compounds of Formula (I) according to the invention can be used as herbicides by themselves, but they are generally formulated into herbicidal compositions using formulation adjuvants, such as carriers, solvents and surface-active agents (SFAs). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types, many of which are known from the Manual on Development and Use of FAO Specifications for Plant Protection Products, 5th Edition, 1999. These include dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, capsule suspensions (CS) and seed treatment formulations. The formulation type chosen in any instance will depend upon the particular purpose envisaged and the physical, chemical and biological properties of the compound of Formula (I).

Dustable powders (DP) may be prepared by mixing a compound of Formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of Formula (I) is present initially in either the water or the solvent/SFA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SFAs), spray additives based on oils, for example certain mineral oils or natural plant oils (such as soy bean and rape seed oil), and blends of these with other bio-enhancing adjuvants (ingredients which may aid or modify the action of a compound of Formula (I)).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

The composition of the present may further comprise at least one additional pesticide. For example, the compounds according to the invention can also be used in combination with other herbicides or plant growth regulators. In a preferred embodiment the additional pesticide is a herbicide and/or herbicide safener. Examples of such mixtures are (in which ‘I’ represents a compound of Formula I). I+acetochlor, I+acifluorfen, I+acifluorfen-sodium, I+aclonifen, I+acrolein, I+alachlor, I+alloxydim, I+ametryn, I+amicarbazone, I+amidosulfuron, I+aminopyralid, I+amitrole, I+anilofos, I+asulam, I+atrazine, I+azafenidin, I+azimsulfuron, I+BCPC, I+beflubutamid, I+benazolin, I+bencarbazone, I+benfluralin, I+benfuresate, I+bensulfuron, I+bensulfuron-methyl, I+bensulide, I+bentazone, I+benzfendizone, I+benzobicyclon, I+benzofenap, I+bicyclopyrone, I+bifenox, I+bilanafos, I+bispyribac, I+bispyribac-sodium, I+borax, I+bromacil, I+bromobutide, I+bromoxynil, I+butachlor, I+butamifos, I+butralin, I+butroxydim, I+butylate, I+cacodylic acid, I+calcium chlorate, I+cafenstrole, I+carbetamide, I+carfentrazone, I+carfentrazone-ethyl, I+chlorflurenol, I+chlorflurenol-methyl, I+chloridazon, I+chlorimuron, I+chlorimuron-ethyl, I+chloroacetic acid, I+chlorotoluron, I+chlorpropham, I+chlorsulfuron, I+chlorthal, I+chlorthal-dimethyl, I+cinidon-ethyl, I+cinmethylin, I+cinosulfuron, I+cisanilide, I+clethodim, I+clodinafop, I+clodinafop-propargyl, I+clomazone, I+clomeprop, I+clopyralid, I+cloransulam, I+cloransulam-methyl, I+cyanazine, I+cycloate, I+cyclosulfamuron, I+cycloxydim, I+cyhalofop, I+cyhalofop-butyl, I+2,4-D, I+daimuron, I+dalapon, I+dazomet, I+2,4-DB, I+I+desmedipham, I+dicamba, I+dichlobenil, I+dichlorprop, I+dichlorprop-P, I+diclofop, I+diclofop-methyl, I+diclosulam, I+difenzoquat, I+difenzoquat metilsulfate, I+diflufenican, I+diflufenzopyr, I+dimefuron, I+dimepiperate, I+dimethachlor, I+dimethametryn, I+dimethenamid, I+dimethenamid-P, I+dimethipin, I+dimethylarsinic acid, I+dinitramine, I+dinoterb, I+diphenamid, I+dipropetryn, I+diquat, I+diquat dibromide, I+dithiopyr, I+diuron, I+endothal, I+EPTC, I+esprocarb, I+ethalfluralin, I+ethametsulfuron, I+ethametsulfuron-methyl, I+ethephon, I+ethofumesate, I+ethoxyfen, I+ethoxysulfuron, I+etobenzanid, I+fenoxaprop-P, I+fenoxaprop-P-ethyl, I+fentrazamide, I+ferrous sulfate, I+flamprop-M, I+flazasulfuron, I+florasulam, I+fluazifop, I+fluazifop-butyl, I+fluazifop-P, I+fluazifop-P-butyl, I+fluazolate, I+flucarbazone, I+flucarbazone-sodium, I+flucetosulfuron, I+fluchloralin, I+flufenacet, I+flufenpyr, I+flufenpyr-ethyl, I+flumetralin, I+flumetsulam, I+flumiclorac, I+flumiclorac-pentyl, I+flumioxazin, I+flumipropin, I+fluometuron, I+fluoroglycofen, I+fluoroglycofen-ethyl, I+fluoxaprop, I+flupoxam, I+flupropacil, I+flupropanate, I+flupyrsulfuron, I+flupyrsulfuron-methyl-sodium, I+flurenol, I+fluridone, I+fluorochloridone, I+fluoroxypyr, I+flurtamone, I+fluthiacet, I+fluthiacet-methyl, I+fomesafen, I+foramsulfuron, I+fosamine, I+glufosinate, I+glufosinate-ammonium, I+glyphosate, I+halauxifen, I+halosulfuron, I+halosulfuron-methyl, I+haloxyfop, I+haloxyfop-P, I+hexazinone, I+imazamethabenz, I+imazamethabenz-methyl, I+imazamox, I+imazapic, I+imazapyr, I+imazaquin, I+imazethapyr, I+imazosulfuron, I+indanofan, I+indaziflam, I+iodomethane, I+iodosulfuron, I+iodosulfuron-methyl-sodium, I+ioxynil, I+isoproturon, I+isouron, I+isoxaben, I+isoxachlortole, I+isoxaflutole, I+isoxapyrifop, I+karbutilate, I+lactofen, I+lenacil, I+linuron, I+mecoprop, I+mecoprop-P, I+mefenacet, I+mefluidide, I+mesosulfuron, I+mesosulfuron-methyl, I+mesotrione, I+metam, I+metamifop, I+metamitron, I+metazachlor, I+methabenzthiazuron, I+methazole, I+methylarsonic acid, I+methyldymron, I+methyl isothiocyanate, I+metolachlor, I+S-metolachlor, I+metosulam, I+metoxuron, I+metribuzin, I+metsulfuron, I+metsulfuron-methyl, I+molinate, I+monolinuron, I+naproanilide, I+napropamide, I+naptalam, I+neburon, I+nicosulfuron, I+n-methyl glyphosate, I+nonanoic acid, I+norflurazon, I+oleic acid (fatty acids), I+orbencarb, I+orthosulfamuron, I+oryzalin, I+oxadiargyl, I+oxadiazon, I+oxasulfuron, I+oxaziclomefone, I+oxyfluorfen, I+paraquat, I+paraquat dichloride, I+pebulate, I+pendimethalin, I+penoxsulam, I+pentachlorophenol, I+pentanochlor, I+pentoxazone, I+pethoxamid, I+phenmedipham, I+picloram, I+picolinafen, I+pinoxaden, I+piperophos, I+pretilachlor, I+primisulfuron, I+primisulfuron-methyl, I+prodiamine, I+profoxydim, I+prohexadione-calcium, I+prometon, I+prometryn, I+propachlor, I+propanil, I+propaquizafop, I+propazine, I+propham, I+propisochlor, I+propoxycarbazone, I+propoxycarbazone-sodium, I+propyzamide, I+prosulfocarb, I+prosulfuron, I+pyraclonil, I+pyraflufen, I+pyraflufen-ethyl, I+pyrasulfotole, I+pyrazolynate, I+pyrazosulfuron, I+pyrazosulfuron-ethyl, I+pyrazoxyfen, I+pyribenzoxim, I+pyributicarb, I+pyridafol, I+pyridate, I+pyriftalid, I+pyriminobac, I+pyriminobac-methyl, I+pyrimisulfan, I+pyrithiobac, I+pyrithiobac-sodium, I+pyroxasulfone, I+pyroxsulam, I+quinclorac, I+quinmerac, I+quinoclamine, I+quizalofop, I+quizalofop-P, I+rimsulfuron, I+saflufenacil, I+sethoxydim, I+siduron, I+simazine, I+simetryn, I+sodium chlorate, I+sulcotrione, I+sulfentrazone, I+sulfometuron, I+sulfometuron-methyl, I+sulfosate, I+sulfosulfuron, I+sulfuric acid, I+tebuthiuron, I+tefuryltrione, I+tembotrione, I+tepraloxydim, I+terbacil, I+terbumeton, I+terbuthylazine, I+terbutryn, I+thenylchlor, I+thiazopyr, I+thifensulfuron, I+thiencarbazone, I+thifensulfuron-methyl, I+thiobencarb, I+topramezone, I+tralkoxydim, I+tri-allate, I+triasulfuron, I+triaziflam, I+tribenuron, I+tribenuron-methyl, I+triclopyr, I+trietazine, I+trifloxysulfuron, I+trifloxysulfuron-sodium, I+trifluralin, I+triflusulfuron, I+triflusulfuron-methyl, I+trihydroxytriazine, I+trinexapac-ethyl, I+tritosulfuron, I+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6). The compounds of the present invention may also be combined with herbicidal compounds disclosed in WO06/024820 and/or WO07/096,576.

The mixing partners of the compound of Formula I may also be in the form of esters or salts, as mentioned e.g. in The Pesticide Manual, Fourteenth Edition, British Crop Protection Council, 2006.

The compound of Formula I can also be used in mixtures with other agrochemicals such as fungicides, nematicides or insecticides, examples of which are given in The Pesticide Manual.

The mixing ratio of the compound of Formula I to the mixing partner is preferably from 1:100 to 1000:1.

The mixtures can advantageously be used in the above-mentioned formulations (in which case “active ingredient” relates to the respective mixture of compound of Formula I with the mixing partner).

The compounds of Formula I according to the invention can also be used in combination with one or more safeners. Likewise, mixtures of a compound of Formula I according to the invention with one or more further herbicides can also be used in combination with one or more safeners. The safeners can be AD 67 (MON 4660), benoxacor, cloquintocet-mexyl, cyprosulfamide (CAS RN 221667-31-8), dichlormid, fenchlorazole-ethyl, fenclorim, fluxofenim, furilazole and the corresponding R isomer, isoxadifen-ethyl, mefenpyr-diethyl, oxabetrinil, N-isopropyl-4-(2-methoxy-benzoylsulfamoyl)-benzamide (CAS RN 221668-34-4). Other possibilities include safener compounds disclosed in, for example, EP0365484 e.g N-(2-methoxybenzoyl)-4-[(methylaminocarbonyl)amino]benzenesulfonamide. Particularly preferred are mixtures of a compound of Formula I with cyprosulfamide, isoxadifen-ethyl, cloquintocet-mexyl and/or N-(2-methoxybenzoyl)-4-[(methyl-aminocarbonyl)amino]benzenesulfonamide.

The safeners of the compound of Formula I may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phos-phonium salt thereof as disclosed in WO 02/34048, and the reference to fenchlorazole-ethyl also applies to fenchlorazole, etc.

Preferably the mixing ratio of compound of Formula I to safener is from 100:1 to 1:10, especially from 20:1 to 1:1.

The mixtures can advantageously be used in the above-mentioned formulations (in which case “active ingredient” relates to the respective mixture of compound of Formula I with the safener).

The present invention still further provides a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2000 g/ha, especially from 50 to 1000 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

In a preferred embodiment the crop plant is rendered tolerant to HPPD-inhibitors via genetic engineering. Methods of rending crop plants tolerant to HPPD-inhibitors are known, for example from WO0246387. Thus in an even more preferred embodiment the crop plant is transgenic in respect of a polynucleotide comprising a DNA sequence which encodes an HPPD-inhibitor resistant HPPD enzyme derived from a bacterium, more particularly from Pseudomonas fluorescens or Shewanella colwelliana, or from a plant, more particularly, derived from a monocot plant or, yet more particularly, from a barley, maize, wheat, rice, Brachiaria, Chenchrus, Lolium, Festuca, Setaria, Eleusine, Sorghum or Avena species.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium. Weeds can also include plants which may be considered crop plants but which are growing outside a crop area (‘escapes’), or which grow from seed left over from a previous planting of a different crop (‘volunteers’). Such volunteers or escapes may be tolerant to certain other herbicides.

The compounds of the present invention can be prepared according to Schemes 1 to 2.

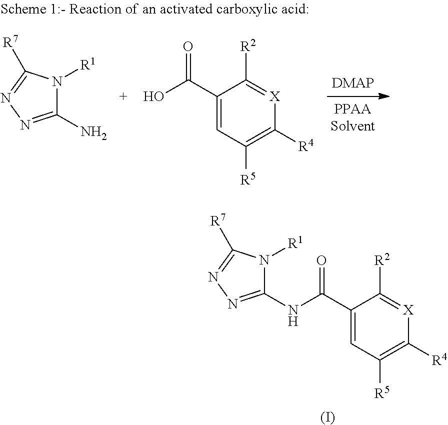

DMAP=4-dimethylaminopyridine, PPAA=1-propanephosphonic acid cyclic anhydride, and the solvent is a non-protic organic solvent such as ethyl acetate.

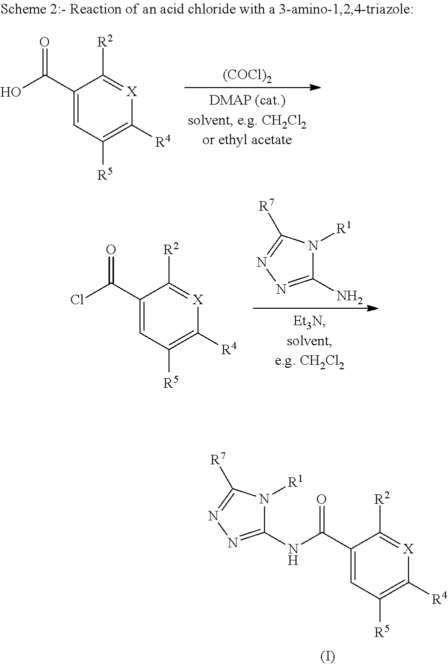

The carboxylic acids are known, or can be prepared by known methods or methods analogous to known methods. N-4-alkylated 3-amino-1,2,4-triazoles can be prepared by the method shown in Scheme 3.

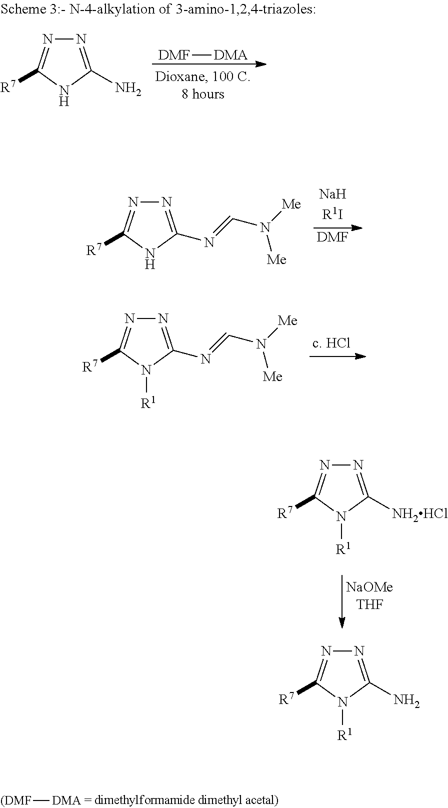

5-Substituted 3-amino-1,2,4-triazoles can be prepared by the method shown in Scheme 4.

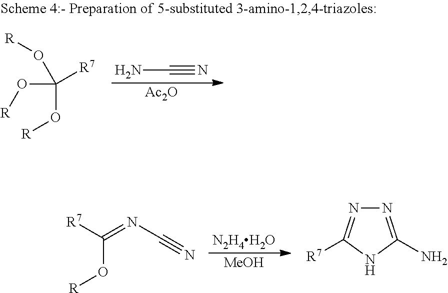

### EXAMPLE P1

### Experimental Procedure for the Preparation of Compound 1.008

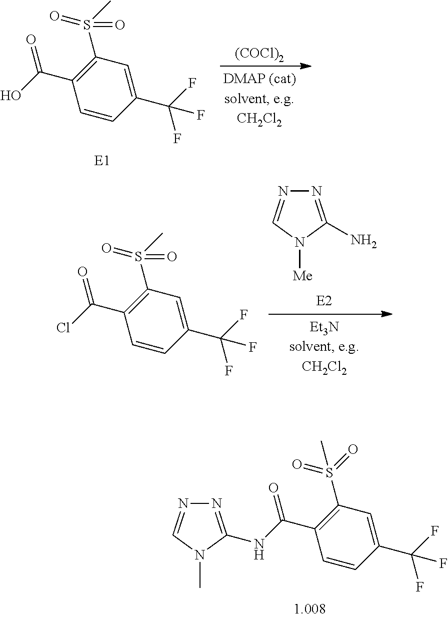 [SMILES: CS(=O)(=O)c1cc(C(F)(F)F)ccc1C(=O)O ; "(COCl)2" ; CS(=O)(=O)c1cc(C(F)(F)F)ccc1C(=O)Cl ; Cn1cnnc1N ; Cn1cnnc1NC(=O)c1ccc(C(F)(F)F)cc1S(C)(=O)=O]

STEP 1: Oxalyl chloride (0.386 ml, 4.47 mmol) was added dropwise to a solution of the benzoic acid E1 (400 mg, 1.49 mmol) in HPLC grade dichloromethane (15 ml), containing a catalytic amount of DMAP. The reaction mixture was allowed to stir at room temperature for 2 hours. The solvent was then removed under reduced pressure to leave the crude benzoyl chloride, which was used without further purification.

STEP 2: The crude benzoyl chloride from step 1 was dissolved in 10 ml HPLC grade dichloromethane, and the solution was cooled to 0 C and the aminotriazole E2 (150 mg, 1.49 mmol) was added. The reaction mixture was then allowed to warm to room temperature, and triethylamine (0.315 ml, 2.23 mmol) was added. Stirring was continued for another 3 hours, then the reaction mixture was diluted with dichloromethane (50 ml) and washed with water (2×20 ml) and brine (1×10 ml). The dichloromethane layer was then dried over sodium sulfate, and evaporated under reduced pressure to afford the crude product. This was purified on a Combiflash, eluting with ethyl acetate-hexane, to afford the pure product as a white solid (90 mg). Yield: 17%

### EXAMPLE P2

### Experimental Procedure for the Preparation of Compound 1.013

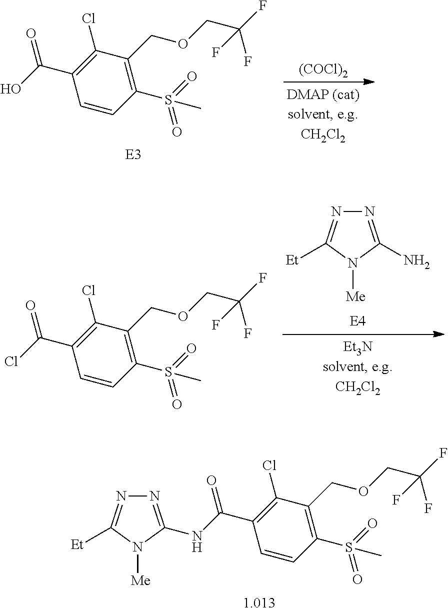

STEP 1: Oxalyl chloride (0.224 ml, 2.60 mmol) was added dropwise to a solution of the benzoic acid E3 (300 mg, 0.867 mmol) in HPLC grade dichloromethane (15 ml), containing a catalytic amount of DMAP. The reaction mixture was allowed to stir at room temperature for 2 hours, and the solvent was then removed under reduced pressure to afford the crude benzoyl chloride, which was used without further purification.

STEP 2: The crude acid chloride from step 1 mass was dissolved in HPLC grade dichloromethane (10 ml) and the solution was cooled to 0 C. The aminotriazole E4 (110 mg, 0.867 mmol) was added. The reaction mixture was then allowed to warm to room temperature, and triethylamine (0.110 ml, 0.867 mmol) was added. Stirring was continued for another 3 hours, then the reaction mixture was diluted with dichloromethane (50 ml) and washed with water (2×20 ml) and brine (1×10 ml). The dichloromethane layer was then dried over sodium sulfate, and evaporated under reduced pressure to afford the crude product. This was purified on a Combiflash, eluting with ethyl acetate-hexane, to afford the pure product as a white solid (110 mg). Yield: 28%

### EXAMPLE P3

### Experimental Procedure for the Preparation of Aminotriazole E2

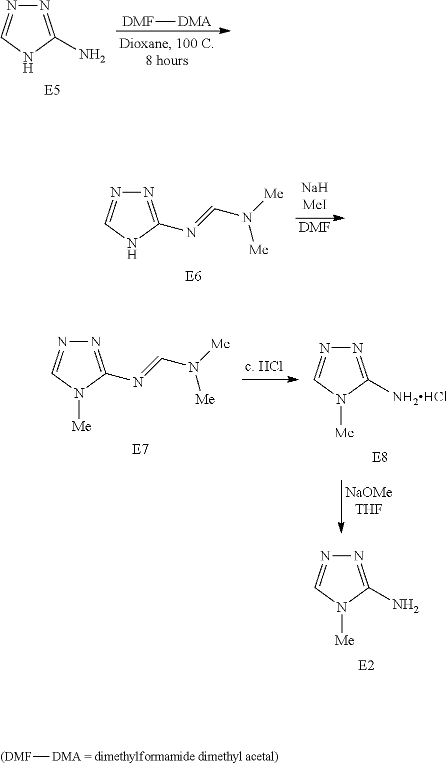

STEP 1: A solution of aminotriazole E5 (10.0 g, 119.05 mmol) in 1,4 dioxane (100 ml) was treated with DMF-DMA (31.62 ml, 238 mmol) at room temperature. The reaction mixture was then heated under reflux for 3 hours, during which time a clear solution inititially formed and then a solid began to form after around 30 min. The reaction mixture was cooled to room temperature, and the solvent and excess DMF-DMA was evaporated under reduced pressure to afford the crude product. This was triturated with diethyl ether-hexane to obtain the pure E6 as an off white solid (8.0 g). Yield: 48%

STEP 2: Sodium hydride (60% in mineral oil, 4.3 g, 107 mmol) was added portionwise to a stirred solution of E6 (15.0 g, 107 mmol) in anhydrous dimethylformamide (150 ml) at 0° C. The reaction mixture was then allowed to stir at room temperature for 1.5 hours, before being cooled to 0° C. and treated dropwise with methyl iodide (10 ml, 161 mmol). The reaction mixture was then stirred at room temperature for 6 hours. The reaction mixture was then diluted with water (300 ml) and extracted with ethyl acetate (3×150 ml). The combined ethyl acetate extracts were dried over sodium sulfate, and the solvent was evaporated under reduced pressure to afford the crude product. This was purified on a Combiflash, eluting with methanol-dichloromethane, to afford pure E7 (5.0 g). Yield: 30%

STEP 3: A stirred solution of E7 (5.0 g, 32.68 mmol) in tetrahydrofuran (50 ml) was treated with conc. hydrochloric acid (5 ml) at room temperature, and was then heated under reflux for 16 hours. The tetrahydrofuran was removed by evaporation under reduced pressure, and the resultant wet solid was dried by azeotroping under reduced pressured with toluene. The dry solid was triturated with diethyl ether-hexane to afford the salt E8 as a white solid (4.0 g). Yield: 93%

STEP 4: Sodium methoxide (1.8 gm, 33 mmol) was added portionwise to a stirred suspension of E8 (4.9 g, 37 mmol) in anhydrous THF (50 ml) at 0° C. Immediately after the addition, the reaction mixture formed a clear solution. This was stirred for a further 2 hours, during which time a solid slowly precipitated out. This was filtered off and dried, yielding the amine E2 as a white solid (0.7 g). Yield: 20%

### EXAMPLE P4

### Experimental Procedure for the Preparation of Aminotriazole E4

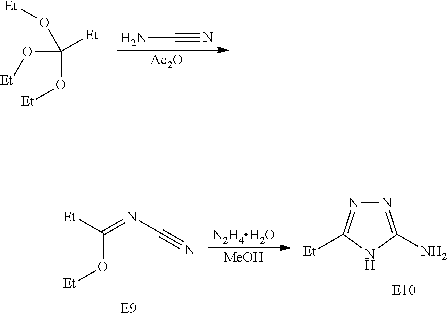

STEP 1: Cyanamide (20 g, 0.476 mmol) was added portionwise to a solution of 1,1,1 trimethoxy propane (84 g, 476 mmol) in acetic anhydride (90 ml) at room temperature. The reaction mixture was then heated at 145 C for 3 hrs. After cooling E9 (65 g) was isolated by distillation. Yield: 63%

STEP 2: Hydrazine hydrate (32 ml, 657 mmol) was added dropwise to a solution of E9 (69 g, 547 mmol) in methanol (300 ml) at room temperature. The reaction mixture was stirred at room temperature for 4 hours, after which the solvent was removed by concentrating under reduced pressure to afford crude E10, which was used in the next step without further purification. Yield: 81%

E10 was then converted to E4 by the procedure described in preparative example P3.

TABLE 1
Examples of herbicidal compounds of the present invention.

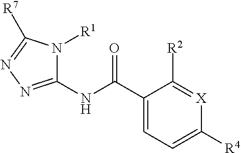

| CMP | R¹ | R⁷ | X | R² | R³ | R⁴ | NMR |
|---|---|---|---|---|---|---|---|
| 1.001 | Me | H | CR³ | Cl | CH₂OCH₂CF₃ | SO₂CH₃ | CDCl3: 3.22 (3H, |
| | | | | | | | s); 3.63 (3H, s); |
| | | | | | | | 4.06 (2H, q); 5.41 |
| | | | | | | | (2H, s); 7.78 (1H, |
| | | | | | | | s); 7.84 (1H, d); |
| | | | | | | | 8.12 (1H, d). |
| 1.002 | Me | H | N | Me | — | CF₃ | |
| 1.003 | Me | H | CR³ | NO₂ | H | SO₂CH₃ | |
| 1.004 | Me | H | N | —CH₂OCH₂CH₂OCH₃ | — | —CF₃ | |
| 1.005 | Me | H | CR³ | Cl | CH₂OCH₂CF₃ | SO₂CH₃ | |
| 1.006 | Me | H | N | Me | — | CF₃ | |
| 1.007 | Me | Me | CR³ | Cl | CH₂OCH₂CF₃ | SO₂CH₃ | CDCl3: 8.11 (1H, |
| | | | | | | | d), 7.82 (1H, d), |
| | | | | | | | 5.41 (2H, s), 4.05 |
| | | | | | | | (2H, q), 3.52 (3H, |
| | | | | | | | s), 3.22 (3H, s), |
| | | | | | | | 2.45 (3H, s). |
| 1.008 | Me | H | CR³ | —S(O)₂CH₃ | H | CF₃ | (DMSO) |
| | | | | | | | 11.56 (broad s, 1H), |
| | | | | | | | 8.35-8.26 (m, 2H), |
| | | | | | | | 8.09 (d, 1H), 7.94 (s, |
| | | | | | | | 1H), 3.81 (s, 3H), |
| | | | | | | | 3.47 (s, 3H) |
| 1.009 | Me | Me | CR³ | —S(O)₂CH₃ | H | CF₃ | (DMSO) 11.46 (s, |
| | | | | | | | 1H), 8.28 (broad s, |
| | | | | | | | 2H), 8.06 (s, 1H), |
| | | | | | | | 3.70 (s, 3H), 3.49 (s, |
| | | | | | | | 3H), 2.22 (s, 3H) |
| 1.010 | Me | Et | CR³ | —S(O)₂CH₃ | H | CF₃ | (DMSO) |
| | | | | | | | 11.46 (broad s, 1H), |
| | | | | | | | 8.34-8.24 (m, 2H), |
| | | | | | | | 8.07 (s, 1H), 3.72 (s, |
| | | | | | | | 3H), 3.48 (s, 3H), |
| | | | | | | | 2.67-2.51 (m, 2H), |
| | | | | | | | 1.21 (t, 3H) |
| 1.011 | Et | H | CR³ | —S(O)₂CH₃ | H | CF₃ | (DMSO) |
| | | | | | | | 11.45 (broad s, 1H), |
| | | | | | | | 8.35-8.24 (m, 2H), |
| | | | | | | | 8.07 (s, 1H), 7.97 (s, |
| | | | | | | | 1H), 4.16 (q, 2H), |
| | | | | | | | 3.47 (s, 3H), 1.36 (t, |
| | | | | | | | 3H) |
| 1.012 | Et | Me | CR³ | —S(O)₂CH₃ | H | CF₃ | (DMSO) 11.33 (s, |
| | | | | | | | 1H), 8.26 (s, 2H), |
| | | | | | | | 8.02 (s, 1H), 4.06 (q, |
| | | | | | | | 2H), 3.48 (s, 3H), |
| | | | | | | | 2.25 (s, 3H), 1.33 (t, |
| | | | | | | | 3H) |
| 1.013 | Me | Et | CR³ | —Cl | CH₂OCH₂CF₃ | SO₂CH₃ | (DMSO) |
| | | | | | | | 11.36 (broad s, 1H), |
| | | | | | | | 8.10 (s, 1H), 7.97 (s, |
| | | | | | | | 1H), 5.25 (s, 2H), |
| | | | | | | | 4.29 (q, 2H), 3.71 (s, |
| | | | | | | | 3H), 3.36 (s, 3H), |
| | | | | | | | 2.68-2.54 (m, 2H), |
| | | | | | | | 1.20 (t, 3H) |
| 1.014 | Et | H | CR³ | —Cl | CH₂OCH₂CF₃ | SO₂CH₃ | (DMSO) |
| | | | | | | | 11.35 (broad s, 1H), |
| | | | | | | | 8.10 (s, 1H), 7.96 (m, |
| | | | | | | | 2H), 5.25 (s, 2H), |
| | | | | | | | 4.30 (q, 2H), 4.12 (q, |
| | | | | | | | 2H), 3.36 (s, 3H), |
| | | | | | | | 1.38 (t, 3H) |
| 1.015 | Et | Me | CR³ | —Cl | CH₂OCH₂CF₃ | SO₂CH₃ | (DMSO) 11.23 (s, |
| | | | | | | | 1H), 8.10 (s, 1H), |
| | | | | | | | 7.95 (s, 1H), 5.25 (s, |
| | | | | | | | 2H), 4.30 (q, 2H), |
| | | | | | | | 4.03 (q, 2H), 3.36 (s, |
| | | | | | | | 3H), 2.24 (broad s, |
| | | | | | | | 3H), 1.34 (broad s, |
| | | | | | | | 3H) |

| | | | | | |
|---|---|---|---|---|---|
| 1.017 | —CH₂CH₂CH₂— | CR³ | —NO₂ | H | SO₂CH₃ |
| 1.018 | —CH₂CH₂CH₂CH₂— | N | CH₂OCH₂CH₂OCH₃ | — | CF₃ |

TABLE 2
Examples of herbicidal compounds of the present invention.

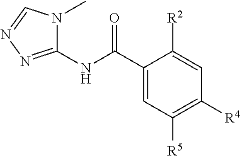

| CMP | R² | R⁴ | R⁵ | NMR |
|---|---|---|---|---|
| 2.001 | CF₃ | H | Cl | (DMSO) 11.39 (s, 1H), 8.03 (s, 1H), 7.88-7.80 |
| | | | | (m, 2H), 3.76 (s, 3H) |
| 2.002 | CF₃ | H | F | (DMSO) 11.38 (s, 1H), 8.02-7.79 (m, 3H), 7.61 (s, |
| | | | | 1H), 3.76 (s, 3H) |
| 2.003 | NO₂ | F | F | (DMSO) 11.51 (s, 1H), 8.47 (broad s, 1H), 8.18 |
| | | | | (broad s, 1H), 7.94 (broad s, 1H), 3.76 (s, 3H) |
| 2.004 | F | Cl | Br | (DMSO) 11.20 (broad s, 1H), 8.19 (d, 1H), |
| | | | | 7.96-7.87 (m, 2H), 3.72 (s, 3H) |
| 2.005 | NO₂ | H | Me | (DMSO) 11.34 (s, 1H), 8.09 (d, 1H), 7.91 (s, 1H), |
| | | | | 7.68 (s, 1H), 7.59 (d, 1H), 3.79 (s, 3H), 2.50 |
| | | | | (s, 3H assumed) |
| 2.006 | CF₃ | H | Me | (DMSO) 11.23 (s, 1H), 7.90 (s, 1H), 7.75 (d, 1H), |
| | | | | 7.65 (s, 1H), 7.55 (d, 1H), 3.74 (s, 3H), 3.46 (s, 3H) |

### BIOLOGICAL EXAMPLES

Seeds of a variety of test species are sown in standard soil in pots Alopecurus myosuroides (ALOMY), Amaranthus retoflexus (AMARE), Setaria faberi (SETFA), Echinochloa crus-galli (ECHCG), Lolium perenne (LOLPE), Solanum nigrum (SOLNI), Stellaria media (STEME) and Digitaria sanguinalis (DIGSA). After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). Compounds are applied at 1000 g/h. The test plants are then grown in a glasshouse under controlled conditions in a glasshouse (at 24/16° C., day/night; 14 hours light; 65% humidity) and watered twice daily. After 13 days for pre and post-emergence, the test is evaluated for the percentage damage caused to the plant. The biological activities are shown in the following table on a five point scale (5=80-100%; 4=60-79%; 3=40-59%; 2=20-39%; 1=0-19%).

| | POST Application | | | | PRE Application | | | |
|---|---|---|---|---|---|---|---|---|
| Compound | AMARE | LOLPE | STEME | DIGSA | AMARE | LOLPE | STEME | DIGSA |
| 1.001 | 5 | 1 | 5 | 5 | 3 | 1 | 1 | 1 |

| | POST Application | | | | | | PRE Application | | | | | |
|---|---|---|---|---|---|---|---|---|---|---|---|---|
| Compound | SOLNI | AMARE | SETFA | ALOMY | ECHCG | IPOHE | SOLNI | AMARE | SETFA | ALOMY | ECHCG | IPOHE |
| 1.008 | 5 | 5 | 3 | 3 | 4 | 5 | 5 | 5 | 3 | 2 | 5 | 4 |
| 1.009 | 4 | 2 | 1 | 2 | 1 | 3 | 2 | 4 | 1 | 1 | 1 | 1 |
| 1.010 | 2 | 1 | 2 | 2 | 2 | 2 | 1 | 1 | 1 | 1 | 1 | 1 |
| 1.011 | 5 | 5 | 2 | 2 | 4 | 5 | 5 | 5 | 2 | 1 | 4 | 4 |
| 1.013 | 2 | 1 | 1 | 2 | 1 | 1 | 1 | 1 | 1 | 1 | 1 | 1 |
| 1.014 | 5 | 5 | 5 | 5 | 5 | 5 | 5 | 5 | 5 | 5 | 5 | 5 |
| 1.015 | 4 | 4 | 1 | 2 | 2 | 4 | 4 | 4 | 1 | 1 | 1 | 2 |
| 2.003 | 1 | 1 | 1 | 1 | 1 | 1 | 1 | 1 | 1 | 1 | 1 | 1 |
| 2.005 | 4 | 4 | 1 | 1 | 1 | 4 | 2 | 2 | 1 | 1 | 1 | 3 |
| 2.006 | 4 | 2 | 1 | 1 | 1 | 2 | 3 | 2 | 1 | 1 | 1 | 3 |

1. A compound of Formula (I): 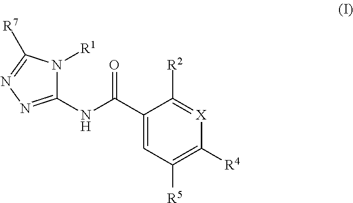
or an agronomically acceptable salt thereof,
wherein:—
R¹ and R⁷ are independently selected from the group consisting of hydrogen, C₁-C₆alkyl, C₁-C₆ haloalkyl and C₁-C₆alkoxy-C₁-C₃alkyl;
or together R¹ and R⁷ form a C₁-C₆ alkylene chain, a C₁-C₆ haloalkylene chain or a C₁-C₃alkyleneoxy-C₁-C₃alkylene chain;
R² is selected from the group consisting of C₁-C₆alkyl-, C₁-C₆haloalkyl-, C₁-C₆alkoxy-C₁-C₆alkyl-, C₁-C₃alkoxy-C₂-C₃alkoxy-C₁-C₃alkyl-, halogen, cyano, nitro, C₁-C₆alkyl-S(O)p- and C₁-C₆haloalkyl-S(O)ₚ—;
X is CR³ or N;
R³ is selected from the group consisting of hydrogen, halo, C₁-C₆alkyl, C₁-C₆haloalkyl, C₁-C₆alkoxy-C₁-C₆alkyl, C₁-C₆haloalkoxy-C₁-C₆alkyl, C₁-C₆alkoxy-C₁-C₆alkoxy-C₁-C₆alkyl, C₁-C₆alkoxy, C₁-C₆haloalkoxy, C₁-C₆alkoxy-C₁-C₆alkoxy, C₁-C₆alkylamino, Cᵣ C₆dialkylamino-, piperidino, morpholino, cyano, C₁-C₆alkyl-S(O)p- and C₁-C₆haloalkyl-S(O)ₚ—;
R⁴ is selected from the group consisting of hydrogen, C₁-C₆alkyl, C₁-C₆haloalkyl, Cᵣ C₆alkoxy-C₁-C₆alkyl, halo, cyano, nitro, C₁-C₆alkyl-S(O)p- and C₁-C₆haloalkyl-S(O)ₚ—;
or R³ and R⁴ together form a saturated 5- or 6-membered ring, optionally containing an oxygen or a S(O)ₚ heteroatom, the 5- or 6-membered ring being optionally substituted by one or more R⁶,
R⁵ is selected from the group consisting of, hydrogen, halogen, C₁-C₆ alkyl and C₁-C₆ haloalkyl;
or R⁴ and R⁵ together form a 5- or 6-membered aromatic ring, optionally containing a nitrogen heteroatom, the 5- or 6-membered aromatic ring being optionally substituted by one or more R⁶;
R⁶ is selected from the group consisting of halo, C₁-C₆alkyl, C₁-C₆haloalkyl, C₁-C₆alkoxy-C₁-C₆alkyl, C₁-C₆haloalkoxy-C₁-C₆alkyl, C₁-C₆alkoxy-C₁-C₆alkoxy-C₁-C₆alkyl-, Cᵣ C₆alkoxy and O₁—C₆haloalkoxy; and
p=0, 1 or 2.

2. A compound according to claim 1, wherein R¹ is methyl or ethyl.

3. A compound according to claim 1, wherein R² is selected from the group consisting of methyl, fluoro, chloro, trifluoromethyl and methyl-S(O)₂—.

4. A compound according to claim 1, wherein X is CR³.

5. A compound according to claim 1, wherein R⁴ is selected from the group consisting of hydrogen, trifluoromethyl and methyl-S(O)₂—.

6. A herbicidal composition comprising a herbicidal compound according to claim 1 and an agriculturally acceptable formulation adjuvant.

7. A herbicidal composition according to claim 6, further comprising at least one additional pesticide.

8. A herbicidal composition according to claim 7, wherein the additional pesticide is a herbicide or herbicide safener.

9. A method of controlling weeds at a locus comprising application to the locus of a weed controlling amount of a composition according to claim 6.

10. (canceled)
